(19) United States
(12) Patent Application Publication
(10) Pub. No.: US 2015/0259319 A1
(43) Pub. Date: Sep. 17, 2015

(54) AROMATIC ALKENOIC ACID DERIVATIVES

(71) Applicant: Symrise AG, Holzminden (DE)

(72) Inventors: Jakob Ley, Holzminden (DE); Katja Obst, Holzminden (DE); Katharina Reichelt, Holzminden (DE); Veronika Somoza, Weidling (AT); Barbara Rohm, Wien (AT)

(21) Appl. No.: 14/644,477

(22) Filed: Mar. 11, 2015

(30) Foreign Application Priority Data
Mar. 12, 2014 (EP) 14 159 306.1

(51) Int. Cl. C07D 317/60; A23L 1/30; A23L 1/29

(52) U.S. Cl. CPC C07D 317/60; A23L 1/293; A23L 1/30; A23V 2002/00

(57) ABSTRACT

What are proposed are primarily aromatic alkenoic acid derivatives of the formula I for use in a therapeutic method as (a) means for reducing appetite and/or (b) means for causing a feeling of satiation and/or as (c) means for reducing energy intake and/or as (d) mood enhancers, and the non-therapeutic use of the aromatic alkenoic acid derivatives of formula I as (a) means for reducing appetite and/or (b) means for causing a feeling of satiation and/or as (c) means for reducing energy intake and/or as (d) mood enhancers.

### FIELD OF THE INVENTION

The invention relates primarily to aromatic alkenoic acid derivatives of the formula I for use in a therapeutic method as (a) means for reducing appetite and/or (b) means for causing a feeling of satiation and/or as (c) means for reducing energy intake and/or as (d) mood enhancers, and to the non-therapeutic use of the aromatic alkenoic acid derivatives of formula I as (a) means for reducing appetite and/or (b) means for causing a feeling of satiation and/or as (c) means for reducing energy intake and/or as (d) mood enhancers.

The invention further relates to a method for (a) reducing the appetite and/or (b) causing a feeling of satiation and/or (c) reducing energy intake and/or (d) enhancing the mood. The invention further encompasses formulations for use in particular therapeutic methods, comprising at least one aromatic alkenoic acid derivative of the formula I and one or more further substances as (a) means for reducing appetite and/or (b) means for causing a feeling of satiation and/or (c) reducing energy intake and/or (d) mood enhancers. The invention also relates to the non-therapeutic use of corresponding formulations. Finally, the invention also relates to orally consumable products (especially foodstuffs, feedstuffs and medicaments) comprising aromatic alkenoic acid derivatives of the formula I, wherein the aromatic alkenoic acid derivative(s) is/are present in a concentration which (a) reduces the appetite and/or (b) causes a feeling of satiation and/or (c) reduces energy intake and/or (d) enhances the mood, but one which is low.

### STATE OF THE ART

The frequent occurrence of lasting excess weight caused by lack of exercise and/or excessive food intake can lead to chronic conditions such as obesity, insulin resistance, lipid metabolism disorders and/or high blood pressure, which have the serious sequelae of type II diabetes, arteriosclerosis, myocardial or cerebral infarction, and hence ultimately lead to early death. More particularly, a high content of readily metabolizable carbohydrates, proteins and particularly fats in food leads to formation of fat depots and can ultimately contribute significantly to the problems mentioned. In order to restrict the intake of these food constituents which are often very preferable for hedonic reasons, particularly the fats and sweet carbohydrates (sugars), the contents thereof in reduced-calorie foodstuffs, called “light products”, are often lowered significantly and replaced by substitutes (for example thickeners for fats, non-calorific sweeteners instead of sugar).

In the case of consumption of “light products”, a product which has a comparable hedonic value to the high-energy original product because of skilful formulation may often be consumed in greater amounts, which then results in the most unfavourable case actually in an increased intake of calorifically relevant food constituents, and hence the aim of lowering the calorific intake is not achieved.

In order to counter increased intake of calorifically relevant food constituents, there has long been a desire to find food constituents, especially aromas which have been assessed as safe, have already been approved and are generally accepted, which can reduce the feeling of hunger and the natural appetite and/or correspondingly increase the feeling of satiation. It is known that, by increasing serotonin secretion in particular areas of the brain, with simultaneous exposure to nutrients through the orally consumable products (especially foodstuffs) consumed, and through induction of leptin receptor and serotonin receptor proteins, it is possible to have a negative effect on the appetite and a positive effect on satiation.

As well as effects on the appetite and satiation, it is also possible to positively influence the mood of the consumer. Thus, there are already known foods and drinks which comprise dopamine and serotonin and have a mood-enhancing effect. If serotonin is available in sufficient amounts in the brain, it causes a positive mood, good concentration capacity and optimism. Low serotonin levels, in contrast, can lead to irritability, sleep disorders, lack of concentration capacity and depression.

Intake of serotonin and dopamine via food increases the serotonin and dopamine concentration in the blood and is very probably also available for interactions with receptors in the brain. In order to increase the serotonin or dopamine concentration in the brain, it is advantageous to stimulate the secretion of serotonin or dopamine in the brain.

The problem addressed by the present invention was therefore that of finding compounds and substances which can make a contribution to reducing appetite and/or to causing a feeling of satiation and/or to reducing energy intake and/or as a mood enhancer, in order to positively influence body health. A further problem addressed by the present invention was that these substances and compounds are not toxic and do not have only restricted usability, such that they can be used in the foods sector. In particular, the substances and compounds should be active even in small amounts and should have a minimum level of unpleasant sensory notes or any secondary taste in products.

### DESCRIPTION OF THE INVENTION

This problem is solved by aromatic alkenoic acid derivatives of the formula I

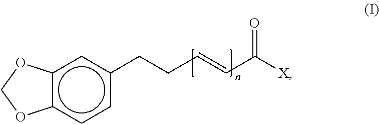

for use in a therapeutic method
(i) for reducing appetite, preferably for reducing calorific intake and in this case preferably for therapeutically reducing weight,
and/or
(ii) for causing a feeling of satiation, preferably for reducing calorific intake and in this case preferably for therapeutically reducing weight,
and/or
(iii) as a mood enhancer,
where
n=1, 2, where the resulting double bond(s) are each independently in E or Z configuration,
X=—OR¹ or —NR²R³, with
R¹=hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl or isoprenyl,
and where
R², R³ are each independently selected from the group consisting of: hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl, isoprenyl,
or
R² and R³ together form a saturated or unsaturated carbocyclic ring selected from the group consisting of: —CH2-CH2-, —CH2-CH2-CH2-, —CH2-CH2-CH2-CH2-, —CH═CH—CH═CH—, —CH2-CH2-CH2-CH2-CH2-, —CH═CH—CH2-CH2-CH2-, —CH2-CH═CH—CH2-CH2-, —CH═CH—CH═CH—CH2-, —CH═CH—CH═CH—CH═.

The present invention likewise relates to the cosmetic use of these aromatic alkenoic acid derivatives of the formula I for reducing appetite, preferably for reducing calorific intake and/or for causing a feeling of satiation, preferably for reducing calorific intake and in this case preferably for cosmetic reduction of bodyweight.

In a preferred embodiment, the aromatic alkenoic acid derivatives of the formula I are used in the treatment of obesity.

A further aspect of the present invention is additionally the non-therapeutic use of aromatic alkenoic acid derivatives of the formula I

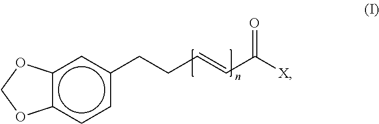

(i) for reducing appetite, preferably for non-therapeutically reducing calorific intake and in this case preferably for non-therapeutically reducing weight, and/or
(ii) for non-therapeutically causing a feeling of satiation, preferably for non-therapeutically reducing calorific intake and in this case preferably for non-therapeutically reducing weight,
and/or
(iii) as a mood enhancer,
where
n=1, 2, where the resulting double bond(s) are each independently in E or Z configuration,
X=—OR¹ or —NR²R³, with
R1=hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl or isoprenyl,
and where
R², R³ are each independently selected from the group consisting of: hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl, isoprenyl,
or
R² and R³ together form a saturated or unsaturated carbocyclic ring selected from the group consisting of: —CH2-CH2-, —CH2-CH2-CH2-, —CH2-CH2-CH2-CH2-, —CH═CH—CH═CH—, —CH2-CH2-CH2-CH2-CH2-, —CH═CH—CH2-CH2-CH2-, —CH2-CH═CH—CH2-CH2-, —CH═CH—CH═CH—CH2-, —CH═CH—CH═CH—CH═.

Preference is given to the following aromatic alkenoic acid derivatives of the formula I:

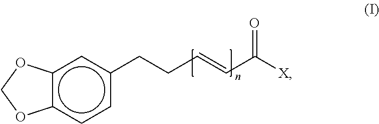

where
n=1, 2, where more than 50% of the resulting double bond(s) are in E configuration,
X=—OR¹ or —NR²R³, with
R¹=methyl, ethyl
R²=hydrogen,
R³=2-methylpropyl, 2-methyl-1-butyl,
or
R² and R³ together form a saturated carbocyclic ring of —CH₂—CH₂—CH₂—CH₂—CH₂—,
in a therapeutic method or in a non-therapeutic use.

Particular preference is given here to the aromatic alkenoic acid derivatives of the formula I selected from the group consisting of:
5-(1,3-benzodioxol-5-yl)-N-isobutylpent-2-enamide (dihydropiperlinguminine, compound 1)

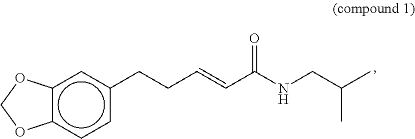

7-(1,3-benzodioxol-5-yl)-N-isobutylhepta-2,4-dienamide (chingchengenamide A, compound 2):

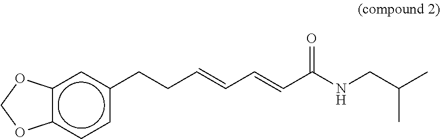

7-(1,3-benzodioxol-5-yl)-1-(1-piperidyl)hepta-2,4-dien-1-one (piperdardine, compound 3)

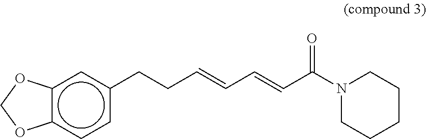

methyl 7-(1,3-benzodioxol-5-yl)hepta-2,4-dienoate (compound 4)

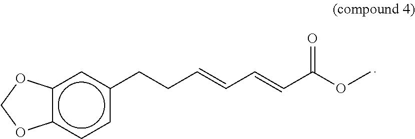

The inventive aromatic alkenoic acid derivatives are in the E configuration preferably to an extent of more than 50%, more preferably to an extent of more than 80%, especially preferably to an extent of more than 90%, where the aromatic alkenoic acid derivatives are preferably selected from compounds 1 to 4.

Preference is given to using the compounds 1 to 4 in cosmetic products for reducing appetite, preferably for reducing calorific intake and/or for causing a feeling of satiation, preferably for reducing calorific intake and in this case preferably for cosmetic reduction of weight.

Likewise preferably, compounds 1 to 4 are used in food products, semi-luxury goods and/or food supplement products for reducing appetite, preferably for reducing calorific intake and/or for causing a feeling of satiation, preferably for reducing calorific intake, preferably for reducing bodyweight.

“Cosmetic” in the present invention is understood to mean the use of cosmetic compositions, i.e. substances or mixtures intended for body care and beauty, or for the conservation, restoration or improvement of the beauty of the human body.

The particularly preferred aromatic alkenoic acid derivatives 1 to 4 mentioned all occur naturally, in particular plant species, some of these plants being usable in foods directly or in the form of extracts and formulations. Compound 1 was found, for example, in Piper longum (long pepper, Tabuneng, W., Bando, H., Amiya, T., 1983. Chemical & Pharmaceutical Bulletin 31, 3562-3565), compound 2 in P. nigrum (Friedman, M., Levin, C. E., Lee, S. U., Lee, J. S., Ohnisi-Kameyama, M., Kozukue, N., 2008. J Agric Food Chem 56, 3028-3036) and compound 3 in Piper nigrum (black pepper, Kapoor, I. P., Singh, B., Singh, G., De Heluani, C. S., De Lampasona, M. P., Catalan, C. A., 2009. J Agric Food Chem 57, 5358-5364). Compound 4 has not been described to date in the literature as a natural product.

Accordingly, a plant extract comprising compound 4 (methyl 7-(1,3-benzodioxol-5-yl)hepta-2,4-dienoate) and compound 4 (methyl 7-(1,3-benzodioxol-5-yl)hepta-2,4-dienoate) likewise form part of the subject-matter of the present invention.

The plant extracts comprise one or more of the preferred aromatic alkenoic acid derivatives mentioned and are likewise usable for the purposes of the invention, for example pepper extract (Piper ssp., especially P. nigrum, P. falconeri, P. boehmeriaefolium, P. tuberculatum, P. sintenensis, P. longum, P. chaba or P. guineense), extracts from Fagara species (Fagara macrophylla) and extracts from Macropiper excelsum.

The plant extracts comprise one or more of the aromatic alkenoic acid derivatives of the formula I, especially of the compounds 1 to 4. The alkenoic acid derivatives of the formula I can be obtained from the corresponding fresh or dried plants or plant parts, but especially from white, green or black peppercorns (P. nigrum), long pepper (P. longum), plant parts from the Fagara species or the leaves, seeds or infructescences of Macropiper excelsum. Typically, the dried plant parts (for example fresh or dried roots, fruits, seeds, bark, wood, stems, leaves, flowers or flower parts), preferably in comminuted form, are extracted with a solvent suitable for foods and semi-luxury goods at temperatures of 0° C. up to the boiling point of the particular solvent or solvent mixture, then filtered, and the filtrate is wholly or partly concentrated, preferably by distillation, freeze-drying or spray-drying. The crude extract thus obtained can then be worked up further, for example subjected to enzyme treatment, or treated with acid (for example under pressure), with acidic ion exchangers or with steam, usually at pressures of 0.01 mbar to 100 bar, preferably at 1 mbar to 20 bar, and/or taken up in a solvent suitable for foods and semi-luxury goods. Preference is given to extracts in which the aromatic alkenoic acid derivatives constitute the majority based on the total amount of the amide-type compounds, for example 1% to 90%, preferably 5% by weight, more preferably 10% by weight, more preferably 50% by weight, most preferably 80% by weight, based on the total amount of the amide-type compounds.

Solvents suitable for extraction, especially for foods and semi-luxury goods, are water, ethanol, methanol, 1,2-propylene glycol, glycerol, acetone, dichloromethane, ethyl acetate, diethyl ether, hexane, heptane, triacetin, vegetable oils or fats, supercritical carbon dioxide and mixtures thereof.

Preferred auxiliaries or carriers are maltodextrin, starch, natural or synthetic polysaccharides and/or plant gums such as modified starches or gum arabic, solvents approved for aroma compositions, for example ethanol, 1,2-propylene glycol, water, glycerol, triacetin, vegetable oil triglycerides, colourants, for example approved food dyes, colouring plant extracts, stabilizers, preservatives, antioxidants, viscosity-influencing substances.

The present invention also relates to a method for non-therapeutic use

(i) for reducing appetite, preferably for non-therapeutically reducing calorific intake and in this case preferably for non-therapeutically reducing weight, and/or
(ii) for non-therapeutically causing a feeling of satiation, preferably for non-therapeutically reducing calorific intake and in this case preferably for non-therapeutically reducing weight, and/or
(iii) for enhancing the mood,
comprising the following step:
administering an amount that (a) reduces the appetite and/or (b) causes a feeling of satiation and/or (c) enhances the mood of one or more aromatic alkenoic acid derivative of the formula I

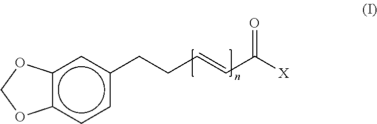

where
n=1, 2, where the resulting double bond(s) are each independently in E or Z configuration,
X=—OR¹ or —NR²R³, with
R¹=hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl or isoprenyl,
and where
R², R³ are each independently selected from the group consisting of: hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl, isoprenyl,
or
R² and R³ together form a saturated or unsaturated carbocyclic ring selected from the group consisting of: —CH2-CH2-, —CH2-CH2-CH2-, —CH2-CH2-CH2-CH2-, —CH═CH—CH═CH—, —CH2-CH2-CH2-CH2-CH2-, —CH═CH—CH2-CH2-CH2-, —CH2-CH═CH—CH2-CH2-, —CH═CH—CH═CH—CH2- or —CH═CH—CH═CH—CH═.

Preference is given here to using the aromatic alkenoic acid derivative compounds 1 to 4, where 1, 2, 3 or all 4 compounds may be present alone or in a mixture.

The aromatic alkenoic acid derivatives of the formula I are preferably incorporated into compositions or formulations which are preferably orally consumable products. Such a composition or formulation preferably comprises at least one, two, three or four aromatic alkenoic acid derivatives of the formula I, preferably compounds 1 to 4.

Accordingly, the present invention further provides a formulation comprising at least one aromatic alkenoic acid derivative of the formula I

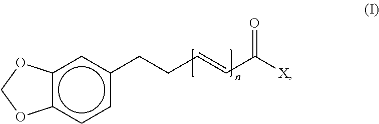

for use in a therapeutic method or in a non-therapeutic use
(i) for reducing appetite, preferably for non-therapeutically reducing calorific intake and in this case preferably for non-therapeutically reducing weight, and/or
(ii) for non-therapeutically causing a feeling of satiation, preferably for non-therapeutically reducing calorific intake and in this case preferably for non-therapeutically reducing weight, and/or
(iii) as a mood enhancer,
where
n=1, 2, where the resulting double bond(s) are each independently in E or Z configuration,
X=—OR¹ or —NR²R³, with
R¹=hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl or isoprenyl,
and where
R², R³ are each independently selected from the group consisting of: hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl, isoprenyl,
or
R² and R³ together form a saturated or unsaturated carbocyclic ring selected from the group consisting of: —CH2-CH2-, —CH2-CH2-CH2-,

### —CH2-CH2-CH2-CH2-, —CH═CH—CH═CH—, —CH2-CH2-CH2-CH2-CH2-, —CH═CH—CH2-CH2-CH2-, —CH2-CH═CH—CH2-CH2-, —CH═CH—CH═CH—CH2-, —CH═CH—CH═CH—CH═.

Preference is given to using, in such a formulation for use in a therapeutic method or in a non-therapeutic use, aromatic alkenoic acid derivatives

where
n=1, 2, where more than 50% of the resulting double bond(s) are in E configuration,
X=—OR1 or —NR2R3, with
R¹=methyl, ethyl
R²=hydrogen,
R³=2-methylpropyl, 2-methyl-1-butyl,
or
R2 and R3 together form a saturated carbocyclic ring consisting of

### —CH2-CH2-CH2-CH2-CH2-,

and
where more than 50% of the aromatic alkenoic acid derivatives are in E configuration.

Preference is given to using, in such a formulation for use in a therapeutic method or in a non-therapeutic use, aromatic alkenoic acid derivatives selected from the group consisting of:
5-(1,3-benzodioxol-5-yl)-N-isobutylpent-2-enamide (compound 1),
7-(1,3-benzodioxol-5-yl)-N-isobutylhepta-2,4-dienamide (compound 2),
7-(1,3-benzodioxol-5-yl)-1-(1-piperidyl)hepta-2,4-dien-1-one (compound 3),
methyl 7-(1,3-benzodioxol-5-yl)hepta-2,4-dienoate (compound 4).

Compounds 1 to 4 here are in E configuration preferably to an extent of more than 50%, more preferably to an extent of more than 80%, especially preferably to an extent of more than 90%.

The particularly preferred aromatic alkenoic acid derivatives 1 to 4 can be obtained as plant extracts, for example as pepper extract (Piper ssp., especially P. nigrum, P. falconeri, P. boehmeriaefolium, P. tuberculatum, P. sintenensis, P. longum, P. chaba or P. guineense), extracts from Fagara species (Fagara macrophylla) and extracts from Macropiper excelsum. As well as these aromatic alkenoic acid derivatives, particular nonaromatic alkamides are also to be found among the plant extracts, especially N-isobutyl-2E,4E-decadienamide (trans-pellitorine). trans-Pellitorine is likewise known as a substance which exhibits satiation-reducing effects. Therefore, the parallel occurrence in the extracts of, for example, Piper nigrum, Piper longum or Macropiper excelsum is highly advantageous, and so a combination of trans-pellitorine with aromatic alkenoic acid derivatives is a preferred embodiment.

Likewise possible is a combination with capsaicinoids, especially with nonivamide (N-nonanoylvanillylamine), which can likewise be used for reducing appetite and/or for causing a feeling of satiation and/or for enhancing the mood.

Therefore, a preferred embodiment of the present invention consists in formulations for use in a therapeutic method or in a non-therapeutic use, wherein the formulation comprises at least one aromatic alkenoic acid derivative of formula I and/or N-isobutyl-2E,4E-decadienamide (trans-pellitorine) and/or nonivamide (N-nonanoylvanillylamine) and/or capsaicin.

In all embodiments, preference is given to individual substances or mixtures of the aromatic alkenoic acid derivatives of the formula I exhibiting only a sub-threshold aroma value in the use concentration, meaning that preference is given to using the compounds around the recognition threshold or lower (based on the ready-to-eat food). This likewise applies to the compounds nonivamide (N-nonanoylvanillylamine) and trans-pellitorine, and also to capsaicinoids, when they are present in such a formulation.

Accordingly, a preferred embodiment is a formulation for use in a therapeutic method or in a non-therapeutic use, wherein the amount of aromatic alkenoic acid derivative(s) comprises N-isobutyl-2E,4E-decadienamide (trans-pellitorine) and/or nonivamide (N-nonanoylvanillylamine) and/or capsaicin.

It has been found that, surprisingly, the inventive aromatic alkenoic acid derivatives of the formula I are capable, even in a concentration range of 0.01 μM to 10 μM (about 0.02 mg/kg to about 2 mg/kg), of triggering serotonin release in neurons by a maximum of up to about 300% against the respective control.

The respective concentrations of the aromatic alkenoic acid derivatives are low compared to typical use concentrations in order to achieve a flavour effect, i.e. strongly perceptible aroma effects are not to be expected.

Accordingly, a preferred embodiment is a formulation for use in a therapeutic method or in a non-therapeutic use, wherein the total concentration of the aromatic alkenoic acid derivatives of the formula I in the formulation is in a region of less than 20 mg/kg, preferably in a concentration of 5 mg/kg or less, more preferably in a concentration of 2 mg/kg or less, based on the total mass of the formulation.

Nonivamide (N-nonanoylvanillylamine) and trans-pellitorine and the inventive aromatic alkenoic acid derivatives of the formula I have equivalent activities for reducing appetite, preferably for non-therapeutically reducing calorific intake and in this case preferably for non-therapeutically reducing weight, and/or for non-therapeutically causing a feeling of satiation, preferably for non-therapeutically reducing calorific intake and in this case preferably for non-therapeutically reducing weight, and/or for enhancing the mood.

The combination of N-isobutyl-2E,4E-decadienamide (trans-pellitorine) and/or nonivamide (N-nonanoylvanillylamine) and/or capsaicin and the inventive aromatic alkenoic acid derivatives of the formula I, especially compounds 1 to 4, individually or in a mixture, is therefore particularly advantageous, since the inventive aromatic alkenoic acid derivatives in particular, at the abovementioned dosage, have a sensorily lower value compared to nonivamide or trans-pellitorine or capsaicinoids, with the same inventive effect. This is especially advantageous since the aromatic alkenoic acid derivatives, especially compounds 1 to 4, are usable in a higher amount in formulations, such as orally consumable products, or can replace some of the nonivamide or some of the trans-pellitorine or of the capsaicinoids, without adversely affecting the flavour, or can even lower the flavour value triggered by nonivamide or trans-pellitorine or capsaicinoids below its threshold value.

Preferably, such a formulation is an orally consumable product or part of an orally consumable product. Such a formulation is especially preferably selected from the group consisting of cosmetics, liquid or solid semi-luxury goods, foodstuffs, semi-finished goods, feedstuffs and medicaments.

Preferably, these inventive aromatic alkenoic acid derivatives are used for inventive use in a therapeutic method or in an inventive non-therapeutic, preferably cosmetic use, in which case the inventive aromatic alkenoic acid derivatives are preferably used alone or as a mixture constituent of an orally consumable product (especially of a cosmetic product, foodstuff, feedstuff or medicament), where the inventive aromatic alkenoic acid derivatives of the formula I are present in a concentration of 20 mg/kg or less, based on the total mass of the orally consumable product.

Preference is given to an orally consumable product in accordance with the invention selected from the group consisting of cosmetic products, semi-luxury goods, foodstuffs (especially liquid or solid, including semi-finished good), feedstuffs and medicaments (pharmaceutical formulations).

The invention accordingly provides a pharmaceutical composition comprising at least one aromatic alkenoic acid derivative of the formula I as active ingredient, preferably at least one or more than one of compounds 1 to 4.

The invention accordingly further provides a cosmetic composition comprising at least one aromatic alkenoic acid derivative of the formula I, preferably at least one or more than one of compounds 1 to 4.

The invention accordingly further provides a semi-luxury good or foodstuff or food supplement comprising at least one aromatic alkenoic acid derivative of the formula I, preferably at least one or more than one of compounds 1 to 4.

In the context of the present text, the term “foodstuff” encompasses a multitude of products. The term “foodstuff” especially includes products as discussed further down in connection with foodstuffs according to the invention.

The term “foodstuff” especially encompasses products which are foods according to DIRECTIVE (EU) No. 178/2002 OF THE EUROPEAN PARLIAMENT AND OF THE COUNCIL dated 28 Jan. 2002. According to this directive, “foods” are all substances or products which are intended to be or can be reasonably expected to be consumed by humans in the processed, partly processed or unprocessed state. “Foods” also include drinks, chewing gum and all substances including water which are deliberately added to the food in the course of production or processing thereof.

The term “feedstuff” in the context of the present text encompasses all kinds of animal nutrition. A multitude of the foods mentioned below can also be used as feedstuffs.

The term “medicament” should be equated with the term pharmaceutical composition or product. It will be apparent that the term “medicament/pharmaceutical composition/pharmaceutical product” in the context of the present text encompasses substances or substance compositions which are intended as compositions having properties for healing or for preventing human or animal diseases, or else can be used in or on the human or animal body or administered to a human or animal, in order either to restore, to correct or to influence the human or animal physiological functions through a pharmacological, immunological or metabolic effect, or to establish a medical diagnosis. Pharmaceutical compositions or products can also be used in the individual case for non-therapeutic, especially cosmetic, purposes.

It will be apparent that foodstuffs or feedstuffs can be converted to corresponding medicaments through the addition substances or substance compositions intended as compositions having properties for healing or for prevention of human or animal diseases.

Preferably, the formulations of the invention include at least 1, 2, 3 or 4 aromatic alkenoic acid derivatives of the formula I, which are preferably selected from the group of compound 1, compound 2, compound 3 and compound 4. It is likewise advantageous when all 4 compounds are present in such a formulation. In the determination of the total concentration of the aromatic alkenoic acid derivatives of a formulation, what is meant is the sum total of the concentrations of the individual compounds.

The present invention accordingly also encompasses orally consumable products comprising at least one, two, three or four aromatic alkenoic acid derivatives of the formula I. Preferably, such an orally consumable product comprises at least 1, at least 2, at least 3 or all four compounds 1 to 4. Preferably, the aromatic alkenoic acid derivatives are in the E configuration to an extent of more than 50%, more preferably to an extent of more than 80%, especially preferably to an extent of more than 90%.

A non-therapeutic use or a non-therapeutic method in the context of the present invention relates to methods which are not intended for surgical or therapeutic treatment of the human or animal body. Preferably, the term “non-therapeutic” in the present invention relates to cosmetic method/cosmetic uses/cosmetic products, and to foodstuffs, semi-luxury goods, feedstuffs and food supplements, in which the inventive aromatic alkenoic acid derivatives, especially compounds 1 to 4, can be included in the formulation in order to reduce appetite or to cause a feeling of satiation or as a mood enhancer, preferably in order to achieve a cosmetic benefit in terms of bodyweight reduction.

In a particularly preferred inventive formulation for use and in inventive non-therapeutic uses of a formulation or in inventive cosmetic uses of a formulation, the formulation comprises N-isobutyl-2E,4E-decadienamide (trans-pellitorine) and/or nonivamide (N-nonanoylvanillylamine) and/or capsaicin.

A further aspect of the present invention relates to the use of a formulation comprising one or more of the aromatic alkenoic acid derivatives mentioned and one or more further substances in a therapeutic method

(i) for reducing appetite, preferably for reducing calorific intake and in this case preferably for therapeutically reducing weight,
and/or
(ii) for causing a feeling of satiation, preferably for reducing calorific intake and in this case preferably for therapeutically reducing weight, and/or
(iii) as a mood enhancer,
wherein, in the therapeutic method, the aromatic alkenoic acid derivative present in the formulation or the aromatic alkenoic acid derivatives present in the formulation is/are used in such a way that it/they (a) reduce appetite and/or (b) cause a feeling of satiation and/or (c) enhance the mood.

In a particularly preferred inventive formulation for use and in particularly preferred inventive non-therapeutic uses of a formulation, and also in particularly preferred inventive cosmetic uses of a formulation, the further substances are preferably spray-dried substances comprising edible constituents, solid carriers and optionally particular aromas or aroma compositions.

In further particularly preferred inventive formulations for use and in particularly preferred inventive uses of a formulation, the further substances are edible solid carriers.

Advantageous carriers in these preferred inventive (preferably spray-dried) formulations are silicon dioxide (silica, silica gel), carbohydrates and/or carbohydrate polymers (polysaccharides), cyclodextrins, starches, degraded starches (starch hydrolysates), chemically or physically modified starches, modified celluloses, gum arabic, ghatti gum, tragacanth, karaya, carrageenan, guar flour, carob flour, alginate, pectins, inulin or xanthan gum. Preferred starch hydrolysates are maltodextrins and dextrins.

Preferred carriers are silicon dioxide, gum arabic and maltodextrins, preference being given here in turn to maltodextrins having DE values in the range from 5 to 20. It is unimportant here what plant originally provided the starch for production of the starch hydrolysates. Suitable and readily available starches are maize-based starches and starches from tapioca, rice, wheat or potatoes. The carriers may also function as flow aids, for example silicon dioxide.

The inventive formulations which, as well as the aromatic alkenoic acid derivative or plurality of aromatic alkenoic acid derivatives for use in accordance with the invention, also comprise one or more solid carriers can be produced, for example, by mechanical mixing operations, in which case there may also be a simultaneous comminution of the particles, or by means of spray-drying. Preference is given to inventive compositions which comprise solid carriers and are produced by means of spray-drying; with regard to spray-drying, reference is made to U.S. Pat. No. 3,159,585, U.S. Pat. No. 3,971,852, U.S. Pat. No. 4,532,145 or U.S. Pat. No. 5,124,162.

Preferred inventive formulations comprising carriers which have been produced by means of spray-drying have a median particle size in the range from 30 to 300 μm and a residual moisture content of less than or equal to 5% by weight.

An inventive formulation is preferably a (preferably spray-dried) formulation comprising

(i) at least one aromatic alkenoic acid derivative of the formula I in an effective amount,
(ii) at least one solid carrier,
(iii) at least one aroma.
The statements made above apply correspondingly with regard to the compounds and mixtures.

The aroma or at least one of the aromas of component (iii) is preferably a sensorily active component and is preferably used in a concentration greater than its stimulus threshold value.

Examples of aromas of component (iii) which may be part of the formulation can be found, for example, in H. Surburg, J. Panten, Common Fragrance and Flavor Materials, 5th ed., Wiley-VCH, Weinheim 2006.

Aromas of component (iii) may be used in the form of aroma compositions which can in turn be used in the form of reaction aromas (Maillard products) and/or extracts or essential oils of plants or plant parts or fractions thereof.

In a further aspect, the present invention relates to orally consumable products comprising at least one aromatic alkenoic acid derivative of the formula I

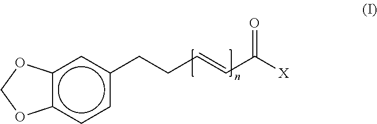

where
n=1, 2, where the resulting double bond(s) are each independently in E or Z configuration,
X=—OR¹ or —NR²R³, with
R¹=hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl or isoprenyl,
and where
R², R³ are each independently selected from the group consisting of: hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl, isoprenyl,
or
R² and R³ together form a saturated or unsaturated carbocyclic ring selected from the group consisting of: —CH2-CH2-, —CH2-CH2-CH2-,

### —CH2-CH2-CH2-CH2-, —CH═CH—CH═CH—, —CH2-CH2-CH2-CH2-CH2-, —CH═CH—CH2-CH2-CH2-, —CH2-CH═CH—CH2-CH2-, —CH═CH—CH═CH—CH2-, —CH═CH—CH═CH—CH═,

where the aromatic alkenoic acid derivative(s) is/are used in a concentration that reduces the appetite and/or causes a feeling of satiation.

The invention further relates to an orally consumable product (especially foodstuff) comprising at least one, preferably two or three or four, aromatic alkenoic acid derivative(s) of the formula I and one or more further substances, where the aromatic alkenoic acid derivative or a mixture of a plurality of aromatic alkenoic acid derivatives is present
in a concentration which reduces appetite and/or causes a feeling of satiation
   and/or
   in a concentration which enhances the mood and simultaneously
in a concentration of 20 mg/kg or less, based on the total mass of the orally consumable product, preferably in a concentration of 5 mg/kg or less, more preferably in a concentration of 2 mg/kg or less, and simultaneously
in a concentration of at least 0.0001 mg/kg or more, based on the total mass of the orally consumable product, preferably in a concentration of 0.001 mg/kg or more, most preferably in a concentration of 0.005 mg/kg or more.

Particular preference is given to an inventive orally consumable product which is a foodstuff, feedstuff and/or medicament.

In order to promote reduction of consumer bodyweight, it has been found to be advisable to limit the potential calorific intake. This can be accomplished firstly by virtue of the inventive use of the aromatic alkenoic acid derivative or a mixture of the aromatic alkenoic acid derivatives in orally consumable products (especially foodstuffs, feedstuffs and medicaments) imparting a feeling of satiation and at the same time reducing appetite, and additionally reducing the absorption of energy-supplying nutrients from the intestine. Not only does this give the consumer reason to limit the eating of energy-containing products, but less dietary energy from the oral energy intake is also available to the consumer through reduced absorption. As well as the lower intake and accumulation of lipids caused by the aromatic alkenoic acid derivatives, the calorific intake can additionally be reduced when inventive orally consumable products (especially foodstuffs, feedstuffs and medicaments) which themselves already have a low energy density are offered for eating.

A preferred inventive orally consumable product (especially foodstuff, feedstuff or medicament) contains not more than 200 kcal/100 g of the orally consumable product, preferably not more than 100 kcal/100 g, more preferably not more than 40 kcal/100 g.

Preferred inventive orally consumable products (especially foodstuffs, feedstuffs and medicaments) are any edible formulations or compositions which serve as food or for oral hygiene or enjoyment, and are regularly products which are intended for introduction into the human or animal oral cavity, to remain therein for a certain period and then either to be eaten (e.g. ready-to-eat foodstuffs or feedstuffs, see also further down), or to be removed again from the oral cavity (for example chewing gum or oral hygiene products or medical mouthwashes). These products include all substances or products which are intended for human or animal intake in the processed, partly processed or unprocessed state. These also include substances which are added to orally consumable products (especially foods, feedstuffs and medicaments) in the course of production or processing thereof, and are intended for introduction into the human or animal oral cavity.

The inventive orally consumable products (especially foodstuffs, feedstuffs and medicaments) also include substances which are intended to be swallowed and then digested by the human or animal in the unaltered, formulated or processed state; in this respect, the inventive orally consumable products also include sheaths, coatings or other enclosures intended to be swallowed as well, or where swallowing is predictable. The term “orally consumable product” encompasses ready-to-consume foodstuffs and feedstuffs, i.e. foodstuffs or feedstuffs already fully assembled in terms of the substances crucial to the taste. The terms “ready-to-eat foodstuff” or “ready-to-eat feedstuff” also include drinks and solid or semisolid ready-to-consume foodstuffs of feedstuffs. Examples include frozen products which have to be thawed before eating and warmed to eating temperature. Products such as yoghurt or ice cream, but also chewing gum or hard caramel, are included among the ready-to-eat foodstuffs or feedstuffs.

Preferred inventive orally consumable products (especially foodstuffs and feedstuffs) also include “semi-finished goods”. A semi-finished good is understood in the context of the present text to mean an orally consumable product which, because of a very high content of aromas and flavourings, is unsuitable for use as a ready-to-eat orally consumable product (especially foodstuff or feedstuff). Only by mixing with at least one further constituent (i.e. by reducing the concentration of the aromas and flavourings in question) and optionally further process steps (e.g. heating, freezing) is the semi-finished product converted to a ready-to-eat orally consumable product (especially foodstuff or feedstuff). Examples of semi-finished products include packet soups, cake flavourings and blancmange powders.

Inventive orally consumable products (especially foodstuffs, feedstuffs and medicaments) also include “oral hygiene products”. An oral hygiene product (also called oral hygiene formulation) in the context of the invention is understood to mean one of the formulations that are familiar to those skilled in the art for cleaning and care of the oral cavity and the pharyngeal cavity, and for freshening the breath. In this context, care of the teeth and gums is explicitly included. Administration forms of standard oral hygiene formulations are especially creams, gels, pastes, foams, emulsions, suspensions, aerosols, sprays, and also capsules, granules, pastilles, tablets, sweets or chewing gum, although this enumeration should not be understood as a limitation for the purposes of this invention.

Preferably, an inventive orally consumable product (especially foodstuff or feedstuff) comprises one or more formulations which serve for nutrition or enjoyment. These especially include (reduced-calorie) bakery products (e.g. bread, biscuits, cakes, other baked goods), confectionery (e.g. chocolates, chocolate bar products, other bar products, fruit gums, sugar-coated sweets, hard and soft caramels, chewing gum), non-alcoholic drinks (e.g. cocoa, coffee, green tea, black tea, (green or black) tea drinks enriched with (green or black) tea extracts, rooibos tea, other herbal teas, fruit-containing lemonades, isotonic drinks, refreshing drinks, nectars, fruit and vegetable juices, fruit or vegetable juice preparations), instant drinks (e.g. instant cocoa drinks, instant tea drinks, instant coffee drinks), meat products (e.g. ham, fresh sausage or raw sausage preparations, spiced or marinated fresh meat or salted meat products), eggs or egg products (dry egg, egg white, egg yolk), cereal products (e.g. breakfast cereals, muesli bars, pre-cooked instant rice products), milk products (e.g. full-fat or reduced-fat or fat-free milk drinks, rice pudding, yoghurt, kefir, fresh cheese, soft cheese, hard cheese, dry milk powder, whey, butter, buttermilk, partly or fully hydrolysed milk protein-containing products), products made from soya protein or other soya bean fractions (e.g. soya milk and products manufactured therefrom, drinks containing isolated or enzymatically treated soya protein, drinks containing soya flour, preparations containing soya lecithin, fermented products such as tofu or tempeh or products manufactured therefrom and mixtures with fruit preparations and optionally aromas), fruit preparations (e.g. jams, fruit ice cream, fruit sauces, fruit fillings), vegetable preparations (e.g. ketchup, sauces, dried vegetables, frozen vegetables, pre-cooked vegetables, parboiled vegetables), snack foods (e.g. baked or fried potato chips or reconstituted potato products, maize- or peanut-based extrudates), fat- and oil-based products or emulsions thereof (e.g. mayonnaise, remoulade, dressings, each full-fat or reduced-fat), other ready meals and soups (e.g. dry soups, instant soups, pre-cooked soups), spices, spice mixtures and especially seasonings which are used, for example, in the snack sector, sweetener preparations, tablets or sachets, other preparations for sweetening or whitening drinks or other foods. The formulations in the context of the invention may also serve as a semi-finished good for production of further formulations which serve as food or for enjoyment. The preparations in the context of the invention may also take the form of capsules, tablets (non-coated and coated tablets, e.g. gastric juice-resistant coatings), sugar-coated tablets, granules, pellets, solid mixtures, dispersions in liquid phases, or of emulsions, powders, solutions, pastes or other swallowable or chewable preparations, or food supplements.

Particular preference is given to reduced-calorie confectionery (e.g. muesli bar products, fruit gums, sugar-coated sweets, hard and soft caramels, chewing gum), non-alcoholic drinks (e.g. cocoa, green tea, black tea, (green or black) tea drinks enriched with (green or black) tea extracts, rooibos tea, other herbal teas, fruit-containing lemonades, isotonic drinks, refreshing drinks, nectars, fruit and vegetable juices, fruit or vegetable juice preparations), instant drinks (e.g. instant cocoa drinks, instant tea drinks), cereal products (e.g. breakfast cereals, muesli bars, pre-cooked instant rice products), milk products (e.g. full-fat or reduced-fat or fat-free milk drinks, rice pudding, yoghurt, kefir, dry milk powder, whey, buttermilk, partly or fully hydrolysed milk protein-containing products), products made from soya protein or other soya bean fractions (e.g. soya milk and products manufactured therefrom, drinks containing isolated or enzymatically treated soya protein, drinks containing soya flour, preparations containing soya lecithin, fermented products such as tofu or tempeh or products manufactured therefrom and mixtures with fruit preparations and optionally aromas), sweetener preparations, tablets or sachets, other preparations for sweetening or whitening drinks or other foods.

Especially preferred are reduced-calorie or calorie-free confectionery (e.g. muesli bar products, fruit gums, sugar-coated sweets, hard caramels, chewing gum), non-alcoholic drinks (e.g. green tea, black tea, (green or black) tea drinks enriched with (green or black) tea extracts, rooibos tea, other herbal teas, fruit-containing low-sugar or sugar-free lemonades, isotonic drinks, nectars, fruit and vegetable juices, fruit or vegetable juice preparations), instant drinks (e.g. instant (green, black, rooibos or herbal) tea drinks), cereal products (e.g. low-sugar or sugar-free breakfast cereals, muesli bars), milk products (e.g. reduced-fat or fat-free milk drinks, yoghurt, kefir, whey, buttermilk), products made from soya protein or other soya bean fractions (e.g. soya milk and products manufactured therefrom, drinks containing isolated or enzymatically treated soya protein, drinks containing soya flour, preparations containing soya lecithin, or products manufactured therefrom and mixtures with fruit preparations and optionally aromas), or sweetener preparations, tablets or sachets.

The preparations may also take the form of capsules, tablets (non-coated and coated tablets, e.g. gastric juice-resistant coatings), sugar-coated tablets, granules, pellets, solid mixtures, dispersions in liquid phases, or of emulsions, powders, solutions, pastes or other swallowable or chewable preparations, for example of food supplements.

The semi-finished goods generally serve for production of ready-to-consume or ready-to-eat formulations which serve as food or for enjoyment.

Further constituents of a ready-to-eat formulation or semi-finished good which serves as food or for enjoyment may be standard base materials, auxiliaries and additives for foods or semi-luxury goods, for example water, mixtures of fresh or processed, vegetable or animal base materials or raw materials, (for example raw, roasted, dried, fermented, smoked and/or cooked meat, bones, cartilage, fish, vegetables, herbs, nuts, vegetable juices or pastes or mixtures thereof), digestible or indigestible carbohydrates (e.g. sucrose, maltose, fructose, glucose, dextrins, amylose, amylopectin, inulin, xylans, cellulose, tagatose), sugar alcohols (e.g. sorbitol, erythritol), natural or hydrogenated fats (e.g. tallow, lard, palm fat, coconut fat, hydrogenated vegetable fat), oils (e.g. sunflower oil, groundnut oil, maize kernel oil, olive oil, fish oil, soya oil, sesame oil), fatty acids or salts thereof (e.g. potassium stearate), proteinogenic or non-proteinogenic amino acids and related compounds (e.g. y-aminobutyric acid, taurine), peptides (e.g. glutathione), native or processed proteins (e.g. gelatin), enzymes (e.g. peptidases), nucleic acids, nucleotides, flavour correctors for unpleasant flavour impressions, further flavour modulators for further, generally not unpleasant flavour impressions, other flavour-modulating substances (e.g. inositol phosphate, nucleotides such as guanosine monophosphate, adenosine monophosphate or other substances such as sodium glutamate or 2-phenoxypropionic acid), emulsifiers (e.g. lecithins, diacylglycerols, gum arabic), stabilizers (e.g. carrageenan, alginate), preservatives (e.g. benzoic acid and salts thereof, sorbic acid and salts thereof), antioxidants (e.g. tocopherol, ascorbic acid), chelators (e.g. citric acid), organic or inorganic acidulants (e.g. acetic acid, phosphoric acid), additional bitter substances (e.g. quinine, caffeine, limonin, amarogentin, humolone, lupolone, catechols, tannins), substances which prevent enzymatic browning (e.g. sulphite, ascorbic acid), essential oils, plant extracts, natural or synthetic dyes or colour pigments (e.g. carotenoids, flavonoids, anthocyanins, chlorophyll and derivatives thereof), spices, trigeminally active substances or plant extracts comprising such trigeminally active substances, synthetic, natural or nature-identical aromas or fragrances, and odour correctors.

Preferably, inventive orally consumable products (especially foodstuffs, feedstuffs and medicaments), for example those in the form of formulations or semi-finished goods, comprise an aroma composition in order to round off and refine the flavour and/or the odour. A formulation may comprise, as constituents, a solid carrier and an aroma composition. Suitable aroma compositions comprise, for example, synthetic, natural or nature-identical aromas, odourants and flavourings, reaction aromas, smoke aromas or other aromatic formulations (e.g. [partial] protein hydrolysates, preferably [partial] protein hydrolysates having a high arginine content, barbecue aromas, plant extracts, spices, spice formulations, vegetables and/or vegetable formulations), and suitable auxiliaries and carriers. In particular, suitable aroma compositions or constituents thereof here are those which can cause a roasted, meaty (especially chicken, fish, seafood, beef, pork, lamb, sheep, goat), vegetable-like (especially tomato, onion, garlic, celery, leek, mushroom, aubergine, seaweed), spicy (especially black and white pepper, cardamom, nutmeg, pimento, mustard and mustard products), fried, yeasty, boiled, fatty, salty and/or pungent aroma impression, and hence enhance the spicy impression. In general, the aroma compositions contain more than one of the ingredients mentioned.

The energy density of an orally consumable product (especially foodstuff, feedstuff or medicament) can be lowered by replacing high-energy ingredients in the orally consumable product with substitutes (e.g. low-calorie thickeners rather than fats, low-calorie or calorie-free sweeteners rather than standard sugar). The disadvantage already discussed above that the consumer will consume an orally consumable product (especially a foodstuff) having reduced energy density in greater amounts, which then results in the most unfavourable case actually in an increased intake of calorifically relevant food constituents, is countered by the use of the aromatic alkenoic acid derivative or a mixture of aromatic alkenoic acid derivatives in orally consumable foodstuffs (especially in foodstuffs). Aromatic alkenoic acid derivative or a mixture of aromatic alkenoic acid derivatives present in an orally consumable product imparts a premature feeling of saturation and simultaneously reduces the appetite and the absorption of lipids. In addition, it has positive effect on the mood of the consumer, which is likewise beneficial.

Preference is given to an inventive orally consumable product selected from the group consisting of confectionery, preferably reduced-calorie or calorie-free confectionery, preferably selected from the group consisting of muesli bar products, fruit gums, sugar-coated sweets, hard caramels and chewing gum,
non-alcoholic drinks, preferably selected from the group consisting of green tea, black tea, (green or black) tea drinks enriched with (green or black) tea extracts, rooibos tea, other herbal teas, fruit-containing low-sugar or sugar-free lemonades, isotonic drinks, nectars, fruit and vegetable juices, fruit and vegetable juice preparations,
instant drinks, preferably selected from the group consisting of instant (green, black, rooibos, herbal) tea drinks,
cereal products, preferably selected from the group consisting of low-sugar and sugar-free breakfast cereals and muesli bars,
milk products, preferably selected from the group consisting of reduced-fat and fat-free milk drinks, yoghurt, kefir, whey, buttermilk and ice cream,
products made from soya protein or other soya bean fractions, preferably selected from the group consisting of soya milk, products manufactured from soya milk, drinks containing isolated or enzymatically treated soya protein, drinks containing soya flour, formulations containing soya lecithin, products manufactured from formulations containing soya lecithin and mixtures with fruit preparations and optionally aromas,
sweetener formulations, tablets and sachets,
sugar-free sweets,
ice cream, with or without milk-based constituents, preferably sugar-free.

Preferably, an inventive orally consumable product (especially foodstuff, feedstuff or medicament) comprises (a) one, two or more sweeteners and/or (b) one, two or more thickeners.

The term “sweeteners” refers here to substances having a relative sweetening power of at least 25, based on the sweetening power of sucrose (which thus has the sweetening power of 1). Sweeteners (a) for use in an inventive orally consumable product (especially foodstuff, feedstuff or medicament) are preferably non-cariogenic and/or have an energy content of not more than 5 kcal per g of the orally consumable product.

Advantageous sweeteners in a preferred inventive orally consumable product (especially foodstuffs, feedstuffs or medicaments) are selected from the following groups (a1) and (a2):

(a1) naturally occurring sweeteners, preferably selected from the group consisting of
(a1-1) miraculin, monellin, mabinlin, thaumatin, curculin, brazzein, pentadin, D-phenylalanine, D-tryptophan, and extracts or fractions which are obtained from natural sources and comprise these amino acids and/or proteins, and the physiologically acceptable salts of these amino acids and/or proteins, especially sodium, potassium, calcium or ammonium salts;
(a1-2) neohesperidin dihydrochalcone, naringin dihydrochalcone, stevioside, steviolbioside, rebaudiosides, especially rebaudioside A, rebaudioside B, rebaudioside C, rebaudioside D, rebaudioside E, rebaudioside F, rebaudioside G, rebaudioside H, rebaudioside M, rebaudioside X, dulcosides and rubusoside, suavioside A, suavioside B, suavioside G, suavioside H, suavioside I, suavioside J, baiyunoside 1, baiyunoside 2, phlomisoside 1, phlomisoside 2, phlomisoside 3 and phlomisoside 4, abrusoside A, abrusoside B, abrusoside C, abrusoside D, cyclocaryoside A and cyclocaryoside I, oslandin, polypodosid A, strogin 1, strogin 2, strogin 4, selligueanin A, dihydroquercetin-3-acetate, perillartine, telosmoside A15, periandrin I-V, pterocaryosides, cyclocaryosides, mukuroziosides, trans-anethole, trans-cinnamaldehyde, bryosides, bryonosides, bryonodulcosides, carnosiflosides, scandenosides, gypenosides, trilobatin, phloridzin, dihydroflavanols, haematoxylin, cyanine, chlorogenic acid, albiziasaponin, telosmosides, gaudichaudioside, mogrosides, mogroside V, hernandulcins, monatin, phyllodulcin, glycyrrhetic acid and derivatives thereof, especially the glycosides thereof such as glycyrrhizin, and the physiologically acceptable salts of these compounds, especially the sodium, potassium, calcium or ammonium salts;
(a1-3) extracts or enriched fractions of the extracts, selected from the group consisting of Thaumatococcus extracts (katamfe plant), extracts from Stevia ssp. (especially Stevia rebaudiana), swingle extracts (Momordica or Siratia grosvenorii, Luo-Han-Guo), extracts from Glycerrhyzia ssp. (especially Glycerrhyzia glabra), extracts from Rubus ssp. (especially Rubus suavissimus), citrus extracts and extracts from Lippia dulcis;
(a2) synthetic sweet-tasting substances, preferably selected from the group consisting of Magap, sodium cyclamate or other physiologically acceptable salts of cyclamic acid, acesulfam K or other physiologically acceptable salts of acesulfam, neohesperidin dihydrochalcone, naringin dihydrochalcone, saccharin, saccharin sodium salt, aspartame, superaspartame, neotame, alitame, advantame, perillartine, sucralose, lugduname, carrelame, sucrononate and sucrooctate.

Advantageous thickeners in a preferred inventive orally consumable product (especially foodstuffs, feedstuffs or medicaments) are selected from the group consisting of: crosslinked polyacrylic acids and derivatives thereof, polysaccharides and derivatives thereof, such as xanthan gum, agar-agar, alginates or tyloses, cellulose derivatives, e.g. carboxymethyl cellulose or hydroxycarboxymethyl cellulose, fatty alcohols, monoglycerides and fatty acids, polyvinyl alcohol and polyvinylpyrrolidone.

Preference is given in accordance with the invention to an orally consumable product (especially foodstuff or feedstuff) which comprises milk thickened by lactic acid bacteria and/or cream thickened by lactic acid bacteria and is preferably selected from the group consisting of orally consumable products having a fat content of 4.0% by weight or less, preferably of 1.5% by weight or less, more preferably 0.5% by weight or less, based in each case on the total weight of the orally consumable product, and/or is selected from the group consisting of yoghurt, kefir and quark.

Preferably, the inventive orally consumable product (especially foodstuff or feedstuff) comprising milk thickened by lactic acid bacteria and/or cream thickened by lactic acid bacteria has an energy content of not more than 150 kcal/100 g of the orally consumable product, preferably not more than 100 kcal/100 g, more preferably not more than 75 kcal/100 g, more preferably not more than 50 kcal/100 g.

A preferred inventive orally consumable product (especially foodstuff or feedstuff) comprising milk thickened by lactic acid bacteria and/or cream thickened by lactic acid bacteria additionally comprises fruit and/or fruit preparations.

Particular preference is given to an inventive orally consumable product (especially foodstuff or feedstuff) comprising milk thickened by lactic acid bacteria and/or cream thickened by lactic acid bacteria, wherein the orally consumable product comprises (i) sugar and/or (ii) thickeners and/or (iii) gelating agents and/or (iv) sweeteners and/or (v) aromas and/or (vi) preservatives.

“Sugar” in the context of the present text (unless stated otherwise or apparent otherwise from the context) is a collective term for all sweet-tasting saccharides (single and double sugars).

Advantageously, an inventive orally consumable product (especially foodstuff or feedstuff) comprising milk thickened by lactic acid bacteria and/or cream thickened by lactic acid bacteria is an orally consumable product comprising a probiotic, where the probiotic is preferably selected from the group consisting of Bifidobacterium animalis subsp. lactis BB-12, Bifidobacterium animalis subsp. lactis DN-173 010, Bifidobacterium animalis subsp. lactis HN019, Lactobacillus acidophilus LA5, Lactobacillus acidophilus NCFM, Lactobacillus johnsonii La1, Lactobacillus casei immunitass/defensis, Lactobacillus casei Shirota (DSM 20312), Lactobacillus casei CRL431, Lactobacillus reuteri (ATCC 55730) and Lactobacillus rham-nosus (ATCC 53013).

Particular preference is given to an inventive orally consumable product (especially foodstuff, feedstock for medicament) which is a chewing gum and contains a chewing gum base. The chewing gum base is preferably selected from the group consisting of chewing gum bases and bubble gum bases. The latter are softer, in order that it is also possible to form bubbles of bubble gum. Chewing gum bases preferred in accordance with the invention include, as well as traditionally used natural resins or the natural latex chicle, elastomers such as polyvinyl acetates (PVA), polyethylenes, (low or medium molecular weight) polyisobutenes (PIBs), polybutadienes, isobutene-isoprene copolymers (butyl rubber), polyvinyl ethyl ethers (PVEs), polyvinyl butyl ethers, copolymers of vinyl esters and vinyl ethers, styrene-butadiene copolymers (styrene-butadiene rubber, SBR) or vinyl elastomers, for example based on vinyl acetate/vinyl laurate, vinyl acetate/vinyl stearate or ethylene/vinyl acetate, and mixtures of the elastomers mentioned, as described, for example, in EP 0 242 325, U.S. Pat. No. 4,518,615, U.S. Pat. No. 5,093,136, U.S. Pat. No. 5,266,336, U.S. Pat. No. 5,601,858 or U.S. Pat. No. 6,986,709. In addition, chewing gum bases for use with preference in accordance with the invention preferably comprise further constituents, for example (mineral) fillers, plasticizers, emulsifiers, antioxidants, waxes, fats or fatty oils, for example hydrogenated vegetable or animal fats, mono-, di- or triglycerides. Suitable (mineral) fillers are, for example, calcium carbonate, titanium dioxide, silicon dioxide, talc, aluminium oxide, dicalcium phosphate, tricalcium phosphate, magnesium hydroxide and mixtures thereof. Suitable plasticizers or detackifiers are, for example, lanolin, stearic acid, sodium stearate, ethyl acetate, diacetin (glycerol diacetate), triacetin (glycerol triacetate), triethyl citrate. Suitable waxes are, for example, paraffin waxes, candelilla wax, carnauba wax, microcrystalline waxes and polyethylene waxes. Suitable emulsifiers are, for example, phosphatides such as lecithin, mono- and diglycerides of fatty acids, e.g. glycerol monostearate.

Inventive chewing gums (especially as disclosed above) preferably comprise constituents such as sugars of various kinds, sugar substitutes, other sweet-tasting substances, sugar alcohols (especially sorbitol, xylitol, mannitol), cooling active ingredients, flavour correctors for unpleasant flavour impressions, further flavour-modulating substances (e.g. inositol phosphate, nucleotides such as guanosine monophosphate, adenosine monophosphate or other substances such as sodium glutamate or 2-phenoxy-propionic acid), humectants, thickeners, emulsifiers, stabilizers, odour correctors and aromas (e.g. eucalyptus menthol, cherry, strawberry, grapefruit, vanilla, banana, citrus, peach, blackcurrant, tropical fruit, ginger, coffee, cinnamon, combinations (of the aromas mentioned) with mint aromas, and spearmint and peppermint alone). Combinations of particular interest include those of the aromas with further substances having cooling, warming and/or mouth-watering properties.

Particular preference is given to an inventive orally consumable product (especially foodstuff, feedstuff or medicament), wherein the orally consumable product is a drink, wherein the drink preferably has a sugar content of 30 g/100 ml of drink or less, preferably of 15 g/100 ml or less, more preferably 5 g/100 ml or less, and more preferably contains no sugar,

and/or
wherein the drink contains no ethanol or contains a maximum of 0.1 percent by volume of ethanol, based on the volume of the drink.

In the context of the present invention, inventive orally consumable products which are ethanol-containing drinks are less preferred.

No ethanol in the present invention means that no ethanol is added, and that the formulation contains less than 0.1% by volume, preferably less than 0.01% by volume, of ethanol, and more preferably does not contain a measurable amount of ethanol.

Particular preference is given to inventive orally consumable products (preferably foodstuffs, feedstuffs or medicaments) which comprise a carbonated drink or a non-carbonated drink.

Further aspects of the present invention are apparent from the examples which follow and the appended claims.

### EXAMPLES

### Preparation Examples 1 to 4

### Compound 1

### 2E-5-(1,3-Benzodioxol-5-yl)-N-isobutylpent-2-enamide (dihydropiperlinguminine)

Compound 1 was isolated from an enriched aqueous ethanolic extract from Macropiper excelsum by preparative HPLC. The spectroscopic data can be found in Table 1.

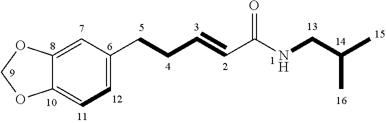

TABLE 1
Spectroscopic data

| | ¹H NMR 400 MHz, CDCl₃ | ¹³C NMR 100 MHz, CDCl₃ |
|---|---|---|
| Position | δ_{H} (ppm), mult (J in Hz) | δ_{C} (ppm) |
| 1 | | 165.8, C |
| 2 | 5.77, dt (1.5, 15.2) | 124.2, CH |
| 3 | 6.84, dt (6.9, 15.2) | 143.3, CH |
| 4 | 2.45, m | 34.1, CH2 |
| 5 | 2.69, m | 34.4, CH2 |
| 6 | | 134.9, C |
| 7 | 6.67, dd (0.4, 1.7) | 108.8, CH |
| 8 | | 147.6, C |
| 9 | 5.92, s | 100.8, CH2 |
| 10 | | 145.8, C |
| 11 | 6.73, dd (0.4, 7.8) | 108.2, CH |
| 12 | 6.62, ddt (0.6, 1.7, 7.9) | 121.1, CH |
| 13 | 3.15, dd (6.1, 6.9) | 46.8, CH2 |
| 14 | 1.79, thept (6.7) | 28.6, CH |
| 15/16 | 0.92, d (6.7) | 20.1, CH3 |

| HR-MS (calc) | HR-MS | [M + H]⁺ | m/z | UV λₘₐₓ (nm) |
|---|---|---|---|---|
| 276.1594 | 276.1588 | C16H22NO3 | 135.0466 | 200, 215, 285 |

### Compound 2

### 2E,4E-7-(1,3-Benzodioxol-5-yl)-N-isobutylhepta-2,4-dienamide (Chingchengenamide A)

Compound 2 was isolated from an enriched aqueous ethanolic extract from Macropiper excelsum by preparative HPLC. The spectroscopic data can be found in Table 2.

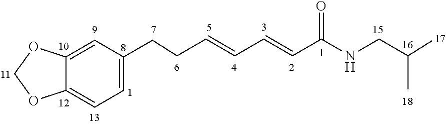 [SMILES: CC(C)CNC(=O)/C=C/C=C/CCc1ccc2c(c1)OCO2]

TABLE 2
Spectroscopic data

| | ¹H NMR 400 MHz, CD₃OD | ¹³C NMR 100 MHz, CD₃OD |
|---|---|---|
| Position | δ_{H} (ppm), mult (J in Hz) | δ_{C} (ppm) |
| 1 | | 169.1, C |
| 2 | 5.91, dt (0.5, 15.2) | 123.4, CH |
| 3 | 7.08, dd (10.5, 15.2) | 142.0, CH |
| 4 | 6.18, ddq (0.7, 1.1, 10.5, 15.2) | 130.3, CH |
| 5 | 6.08, dt (6.7, 15.2) | 142.8, CH |
| 6 | 2.43, q (6.8) | 36.1, CH2 |
| 7 | 2.66, t (7.6) | 36.0, CH2 |
| 8 | | 136.6, C |
| 9 | 6.69, p (0.5, 1.8) | 109.8, CH |
| 10 | | 149.0, C |
| 11 | 5.88, s | 102.0, CH2 |
| 12 | | 147.2, C |
| 13 | 6.70, d (7.9) | 109.0, CH |
| 14 | 6.63, ddt (0.5, 1.8, 7.9) | 122.3, CH |
| 15 | 3.06, d (6.9) | 48.0, CH2 |
| 16 | 1.79, thept (6.9) | 29.8, CH |
| 17/18 | 0.91, d (6.7) | 20.5, CH3 |

| HR-MS (calc) | HR-MS | [M + H]⁺ | m/z | UV λₘₐₓ (nm) |
|---|---|---|---|---|
| 302.1751 | 302.1737 | C18H24NO3 | 135.0454 | 200, 220, 260 |

### Compound 3

### 2E,4E-7-(1,3-Benzodioxol-5-yl)-1-(1-piperidyl)hepta-2,4-dien-1-one (piperdardine)

Compound 3 was isolated from an enriched aqueous ethanolic extract from Macropiper excelsum by preparative HPLC. The spectroscopic data can be found in Table 3.

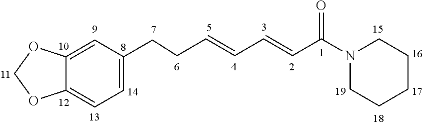 [SMILES: O=C(/C=C/C=C/CCc1ccc2c(c1)OCO2)N1CCCCC1]

TABLE 3
Spectroscopic data

| | ¹H NMR 400 MHz, CD₃OD | ¹³C NMR 100 MHz, CD₃OD |
|---|---|---|
| Position | δ_{H} (ppm), mult (J in Hz) | δ_{C} (ppm) |
| 1 | | 167.8, C |
| 2 | 6.43, d (14.8) | 119.9, CH |
| 3 | 7.12, dd (10.8, 14.8) | 144.4, CH |
| 4 | 6.26, dd (10.8, 15.2) | 130.8, CH |
| 5 | 6.09, dt (6.9, 15.2) | 142.9, CH |
| 6 | 2.43, q (7.3) | 36.1, CH2 |
| 7 | 2.66, t (7.5) | 36.0, CH2 |
| 8 | | 136.5, C |
| 9 | 6.69, d (1.7) | 109.8, CH |
| 10 | | 149.0, C |
| 11 | 5.88, s | 102.0, CH2 |
| 12 | | 147.2, C |
| 13 | 6.70, d (7.9) | 109.0, CH |
| 14 | 6.63, dd (1.7, 7.8) | 122.3, CH |
| 15 | 3.58, m | 44.5, 48.1 CH2 |
| 16 | 1.57, m | 26.9, 27.8, CH2 |
| 17 | 1.69, p (5.6) | 25.5, CH2 |
| 18 | 1.57, m | 26.8, 27.8, CH2 |
| 19 | 3.58, m | 44.5, 48.1 CH2 |

| HR-MS (calc) | HR-MS | [M + H]⁺ | m/z | UV λₘₐₓ (nm) |
|---|---|---|---|---|
| 314.1751 | 314.1752 | C19H24NO3 | 135.0451 | 200, 265 |

### Compound 4

### 2E,4E-Methyl 7-(1,3-benzodioxol-5-yl)hepta-2,4-dienoate

Compound 4 was isolated from an enriched aqueous ethanolic extract from Macropiper excelsum by preparative HPLC. The spectroscopic data can be found in Table 4.

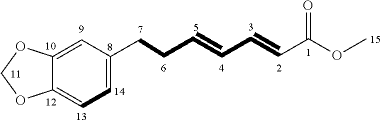

TABLE 4
Spectroscopic data

| | ¹³C NMR 100 MHz, CD₃OD | ¹H NMR 400 MHz, CD₃OD |
|---|---|---|
| Position | δ_{C} (ppm) | δ_{H} (ppm), mult (J in Hz) |
| 1 | 169.4, C | |
| 2 | 119.9, CH | 5.81, d (15.4) |
| 3 | 146.7, CH | 7.23, dd (9.9, 15.4) |
| 4 | 130.1, CH | 6.24, dd (9.9, 15.0) |
| 5 | 145.1, CH | 6.18, dt (6.4, 15.0) |
| 6 | 35.8, CH2 | 2.45, m |
| 7 | 36.2, CH2 | 2.67, t (7.6) |
| 8 | 136.4, C | |
| 9 | 109.8, CH | 6.69, d (1.7) |
| 10 | 149.1, C | |
| 11 | 102.0, CH2 | 5.88, s |
| 12 | 147.2, C | |
| 13 | 109.0, CH | 6.70, d (8.0) |
| 14 | 122.3, CH | 6.63, dd (1.7, 7.8) |
| 15 | 52.0, CH3 | 3.71, s |

| HR-MS (calc) | HR-MS | [M + H]⁺ | m/z | UV λₘₐₓ (nm) |
|---|---|---|---|---|
| 261.1121 | 261.1112 | C15H17O4 | 128.0628, | 200, 260 |
| | | | 135.0451 | |

### Example 5

### Determination of Serotonin Secretion in Neuroblastoma Cells by Compounds 3 and 4

The cell model used for the secretion of the neurotransmitter serotonin is human neuroblastoma cells (SH-SY5Y, ATCC number CRL-2266). They are cultivated at 37° C. and 5% CO2 content with a mixture consisting of equal portions of Eagle's Minimum Essential Medium (MEM) and F12 medium (each with 10% FBS and 1% penicillin/streptomycin). After serotonin release, the cells are harvested with trypsin and, after a vitality test by trypan blue staining, sown in a defined number of cells in 35 mm cell culture dishes.

### Example 6

### Release of Serotonin in the Experimental Cell System with Compounds 1 to 4

After stimulation of 1.25*10⁶ human neuroblastoma cells (SH SY5Y) with 300 μl of Krebs-Ringer HEPES buffer for 3 minutes, 0.1% ascorbic acid, pH 6.2, with or without addition of one of compounds 3 and 4, with adjustment of addition of the compound in concentrations of 0.01 μM, 0.1 μM, 1 μM and 10 μM in the Krebs-Ringer HEPES buffer, the serotonin content is ascertained with the aid of an enzyme-based detection method (Serotonin-ELISA sensitiv, DLD Diagnostica, Hamburg, Germany). The cells are lysed with a sodium laurylsarcosinate-containing buffer and the DNA content is ascertained with the aid of a NanoQuant plate (Tecan, Ménnendorf, Switzerland) for standardization of the serotonin release.

TABLE 5
Serotonin release of SH-SY5Y cells after stimulation
with various concentrations of compounds 3 and 4.

| | Serotonin release | Standard deviation |
|---|---|---|
| Test substance | [% control] | [%] |
| EtOH control (compound 3) | 100 | 34.3 |
| Compound 3, 0.01 μM | 161 | 69.8 |
| Compound 3, 0.1 μM | 141 | 47.6 |
| Compound 3, 1 μM | 283 | 79.8 |
| Compound 3, 10 μM | 226 | 125 |
| Compound 4, 0.01 μM | 129 | 82.1 |
| Compound 4, 0.1 μM | 123 | 72.1 |
| Compound 4, 1 μM | 239 | 113 |
| Compound 4, 10 μM | 171 | 29.7 |

Table 5 shows the influence of compounds 3 and 4 on serotonin release in SH-SY5Y cells. A concentration of 1 μM of compound 3 shows a 280% rise, and a concentration of 1 μM of compound 4 a 240% rise, in serotonin release compared to the control.

### Use Examples

### Use Example 7

### Refreshing Drinks (Sugar-Containing, Reduced-Calorie, Calorie-Free)

The ingredients were mixed in the sequence specified and made up to 100% with water. The mixtures are dispensed into glass bottles and carbonated. The composition is shown in Table A.

TABLE A
Composition of refreshing drink

| | Amount used in % by weight | | | | | | |
|---|---|---|---|---|---|---|---|
| Ingredient | A | B | C | D | E | F | G |
| Sugar (sucrose) | 10 | 10 | 7 | — | — | 8 | 7 |
| Glucose/fructose syrup from | — | — | — | — | 10 | — | — |
| maize, containing 55% by | | | | | | | |
| weight fructose | | | | | | | |
| Rebaudioside A 95% | — | — | 0.02 | 0.05 | — | — | — |
| Citric acid | 0.15 | 0.15 | 0.06 | 0.15 | 0.15 | 0.15 | 0.15 |
| Phosphoric acid | — | — | 0.07 | — | — | — | — |
| Caramel colour | — | — | 0.14 | — | — | — | — |
| Caffeine | — | — | 0.01 | — | — | — | — |
| Lemon aroma | 0.1 | 0.05 | — | 0.1 | 0.1 | 0.1 | 0.1 |
| Lime aroma | — | 0.05 | — | — | — | — | — |
| “Cola”-type drinks emulsion | — | — | 0.05 | — | — | — | — |
| Phloretin | — | — | 0.002 | 0.003 | — | 0.002 | 0.001 |
| Hesperetin | — | — | 0.001 | 0.002 | — | — | 0.002 |
| Extract from Rubus | — | — | — | — | — | 0.01 | — |
| suavissimus, containing 5% by | | | | | | | |
| weight of rubusoside based | | | | | | | |
| on the total weight of the | | | | | | | |
| extract | | | | | | | |
| Homoeriodictyol sodium salt | — | — | 0.005 | — | 0.005 | — | 0.005 |
| trans-Pellitorine | — | — | — | — | — | 0.0001 | 0.0001 |
| Compound 1 | — | — | — | 0.0005 | — | 0.0002 | 0.0001 |
| Compound 2 | — | — | 0.0002 | — | — | — | 0.0001 |
| Compound 3 | 0.0002 | 0.0001 | — | 0.0005 | 0.0001 | 0.0002 | 0.0001 |
| Compound 4 | — | 0.0001 | 0.0002 | — | — | — | 0.0001 |

| | |
|---|---|
| Water | Ad 100 |

### Use Example 8

### Use in a Chewing Gum

Parts I to IV are mixed and kneaded vigorously. The raw material can be processed, for example, in the form of thin strips to give ready-to-eat chewing gums. The composition is shown in Table B.

TABLE B
Composition of chewing gum

| | | Amount used in % by weight | | | | |
|---|---|---|---|---|---|---|
| Part | Ingredient | A | B | C | D | E |
| I | Chewing gum base, “Jagum T” company | 30.495 | 30.495 | 30.4948 | 30.495 | 30.495 |
| II | Sorbitol, pulverized | 39.00 | 39.00 | 39.00 | 39.00 | 39.00 |
| | Isomalt ® (Palatinit GmbH) | 9.50 | 9.50 | 9.50 | 9.50 | 9.50 |
| | Xylitol | 2.00 | 2.00 | 2.00 | 2.00 | 2.00 |
| | Mannitol | 3.00 | 3.00 | 3.00 | 3.00 | 3.00 |
| | Aspartame ® | 0.10 | 0.10 | 0.10 | 0.10 | 0.10 |
| | Acesulfame ® K | 0.10 | 0.10 | 0.10 | 0.10 | 0.10 |
| | Emulgum ® (Colloides Naturels, Inc.) | 0.30 | 0.30 | 0.30 | 0.30 | 0.30 |
| III | Sorbitol, 70% | 14.00 | 14.00 | 14.00 | 14.00 | 14.00 |
| | Glycerol | 1.00 | 1.00 | 1.00 | 1.00 | 1.00 |
| IV | Peppermint aroma | 0.5 | 0.5 | — | 0.5 | 0.5 |
| | Cinnamon fruit aroma | — | — | 0.5 | — | — |
| | trans-Pellitorine | — | — | 0.0002 | — | — |
| | Nonivamide | — | — | — | 0.00001 | 0.00001 |
| | Compound 1 | — | — | 0.0025 | — | — |
| | Compound 2 | — | — | 0.0025 | — | 0.0050 |
| | Compound 3 | 0.0050 | — | 0.0025 | 0.0025 | — |
| | Compound 4 | — | 0.0050 | 0.0025 | 0.0025 | 0.0050 |

### Use Example 9

### Use in Hard Caramels

Palatinitol or the sugar, if appropriate after the addition of the citric acid, was mixed with water and the mixture was melted at 165° C. and then cooled down to 115° C. The aroma and the other constituents were added and, after mixing, poured into moulds, solidified and then removed from the moulds and then packed individually. The composition is shown in Table C.

TABLE C
Composition of hard caramels

| | Content (% by weight) | | | | |
|---|---|---|---|---|---|
| Ingredient | A | B | C | D | E |
| Sugar | 74.50 | — | — | — | — |
| Palatinitol, type | — | 74.00 | 75.50 | 75.00 | 75.00 |
| M | | | | | |
| Citric acid | 0.5 | 1.0 | 0.5 | — | — |
| Yellow dye | — | 0.01 | — | — | — |
| Red dye | — | — | 0.01 | — | — |
| Blue dye | 0.01 | — | — | 0.01 | 0.01 |
| Peppermint aroma | 0.1 | — | — | 0.1 | 0.1 |
| Lemon aroma | — | 0.1 | — | — | — |
| Red fruit aroma | — | — | 0.1 | — | — |
| Rebaudioside A | — | 0.040 | — | 0.040 | 0.040 |
| 98% | | | | | |
| Balansin A | — | 0.005 | 0.010 | 0.005 | — |
| according to | | | | | |
| WO2012164062 | | | | | |
| Hesperetin | — | 0.001 | — | 0.001 | 0.001 |
| Phloretin | — | 0.002 | — | — | — |
| trans-Pellitorine | — | — | 0.00002 | 0.00002 | — |
| Nonivamide | — | — | 0.000005 | — | 0.00001 |
| Compound 1 | 0.0002 | 0.0001 | — | — | — |
| Compound 2 | — | 0.0001 | — | — | |
| Compound 3 | 0.0002 | 0.0001 | — | 0.0001 | 0.0002 |
| Compound 4 | — | 0.0001 | 0.0002 | 0.0001 | — |
| Water | ad 100 | ad 100 | ad 100 | ad 100 | ad 100 |

### Use Example 10

### Low-Fat Yoghurts

The ingredients were mixed and cooled to 5° C. The composition is shown in Table D.

TABLE D
Composition of yoghurts

| | Content in % by weight | | | |
|---|---|---|---|---|
| Ingredient | A | B | C | D |
| Sucrose | 10 | 8 | 6 | — |
| Rebaudioside A 98% | — | — | — | 0.050 |
| Extract from Rubus suavissimus, | — | 0.010 | 0.010 | — |
| containing 5% by weight of | | | | |
| rubusoside based on the total | | | | |
| weight of the extract, for | | | | |
| example of plant extract | | | | |
| Hesperetin | — | 0.001 | 0.001 | 0.002 |
| Phloretin | — | — | 0.002 | 0.002 |
| Homoeriodictyol sodium salt | — | — | — | 0.005 |
| Compound 1 | — | — | — | 0.0002 |
| Compound 2 | — | — | 0.00025 | — |
| Compound 3 | 0.0005 | 0.00025 | — | 0.0002 |
| Compound 4 | — | 0.00025 | 0.0004 | 0.0002 |
| Nonivamide | — | — | — | 0.00001 |

| | |
|---|---|
| Yoghurt, 0.1% fat | make up to 100% |

### Use Example 11

### Fruit Gums

TABLE E
Composition of fruit gums

| | Formulation (figures in % by weight) | | | |
|---|---|---|---|---|
| Ingredient | A | B | C | D |
| Sucrose | 34.50 | 8.20 | 8.20 | 34.50 |
| Glucose syrup, DE 40 | 31.89 | 30.09 | 30.09 | 31.89 |
| Iso Syrup C* Tru Sweet 01750 | 1.50 | 2.10 | 2.10 | 1.50 |
| (Cerestar GmbH) | | | | |
| Gelatine 240 Bloom | 8.20 | 9.40 | 9.40 | 8.20 |
| Polydextrose (Litesse ® Ultra, | — | 24.40 | 24.40 | — |
| Danisco Cultor GmbH) | | | | |
| Yellow and red dye | 0.01 | 0.01 | 0.01 | 0.01 |
| Citric acid | 0.20 | | | 0.20 |
| Cherry aroma, containing 1% by | — | 0.10 | 0.10 | — |
| weight of hesperetin 2 and 0.3% | | | | |
| by weight of phloretin, based | | | | |
| on the aroma | | | | |
| trans-Pellitorine | — | — | 0.00002 | — |
| Nonivamide | — | — | 0.000005 | — |
| Compound 1 | — | 0.0001 | 0.0002 | — |
| Compound 2 | — | — | — | 0.0002 |
| Compound 3 | 0.0002 | 0.0001 | — | 0.0002 |
| Compound 4 | — | 0.0001 | 0.0002 | — |

Polydextrose is a non-sweet-tasting polysaccharide having a low calorific value.

### Use Example 12

### Fruit Juices and Fruit Juice Drinks

TABLE F
Composition of fruit juice

| | Amount used in % by weight | | | | | | |
|---|---|---|---|---|---|---|---|
| Ingredient | A | B | C | D | E | F | G |
| Apple juice | ad 100 | — | — | — | ad 100 | — | ad 100 |
| Apple juice concentrate 10x | — | 10 | — | 10 | — | 10 | — |
| Recovery aroma made from | — | 0.1 | — | 0.1 | — | 0.1 | — |
| apple juice concentrate | | | | | | | |
| Orange juice concentrate 20x | — | — | 5 | — | — | — | — |
| Recovery aroma made from | — | — | 0.05 | — | — | — | — |
| orange juice concentrate | | | | | | | |
| Water | — | — | ad 100 | ad 100 | — | ad 100 | — |
| Ascorbic acid | 0.05 | 0.05 | 0.05 | — | — | — | — |
| trans-Pellitorine | — | — | — | — | — | 0.0001 | — |
| Nonivamide | — | — | — | — | 0.00005 | 0.00005 | 0.00005 |
| Compound 1 | — | — | — | 0.0002 | 0.0001 | — | 0.0002 |
| Compound 2 | — | — | 0.0002 | — | — | 0.0001 | — |
| Compound 3 | 0.0002 | 0.0001 | — | 0.0002 | 0.0001 | 0.0002 | 0.0002 |
| Compound 4 | — | 0.0001 | 0.0002 | — | — | — | — |

### Use Example 13

### Powdered Milkshake

The ingredients are mixed and packed under protective gas or in a vacuum sachet. For use, the powder is stirred up in 100 ml of water (room temperature) and then drunk. The composition is shown in Table G.

TABLE G
Composition of milkshake

| | Amount used in % by weight | | | | | | |
|---|---|---|---|---|---|---|---|
| Ingredient | A | B | C | D | E | F | G |
| Sugar, fine | 5 | 5 | 5 | 5 | 5 | 5 | 5 |
| Nonivamide, 0.1% | — | — | 0.03 | 0.03 | — | 0.015 | — |
| in alcohol | | | | | | | |
| trans-Pellitorine, 1% | — | 0.03 | — | 0.03 | — | — | 0.01 |
| in 1,2-propylene | | | | | | | |
| glycol/ | | | | | | | |
| diethylmalonate | | | | | | | |
| Compound 3, 1% | 0.03 | — | 0.03 | 0.03 | 0.05 | 0.03 | 0.03 |
| in alcohol | | | | | | | |
| Compound 4, 1% | — | 0.03 | 0.03 | 0.01 | 0.02 | — | 0.03 |
| in alcohol | | | | | | | |
| Cappuccino-type | 0.2 | 0.2 | 0.2 | 0.2 | — | — | — |
| aroma, spray-dried | | | | | | | |
| Strawberry-type | — | — | — | — | 0.1 | 0.1 | 0.1 |
| aroma, spray-dried | | | | | | | |
| Beetroot-type colour | — | — | — | — | 0.1 | 0.08 | 0.1 |
| powder, spray-dried | | | | | | | |
| Caramel colour | 0.15 | 0.15 | 0.15 | 0.15 | — | — | — |
| Xanthan | 0.1 | 0.1 | 0.1 | 0.1 | 0.1 | 0.1 | 0.1 |
| Skimmed milk | 10 | 10 | 10 | — | 10 | — | — |
| powder | | | | | | | |
| Lactose-reduced | — | — | — | — | — | 10 | 10 |
| milk powder | | | | | | | |
| (from Omira, | | | | | | | |
| Ravensburg) | | | | | | | |
| Lupine protein | — | — | — | 5 | — | — | — |
| powder | | | | | | | |

1. An aromatic alkenoic acid derivative of formula I 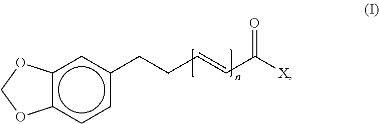
for use in a therapeutic method or non-therapeutic use
(i) for reducing appetite, and/or
(ii) for causing a feeling of satiation, and/or
(iii) as a mood enhancer, wherein
n=1, 2, wherein the resulting double bond(s) are each independently in E or Z configuration,
X=—OR¹ or —NR²R³, with
R¹=hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl or isoprenyl, and
R², R³ are each independently selected from the group consisting of: hydrogen, methyl, ethyl, propyl, 2-propyl, 1-butyl, 2-butyl, 2-ethyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl, isoprenyl, or
R² and R³ together form a saturated or unsaturated carbocyclic ring selected from the group consisting of: —CH₂—CH₂—, —CH₂—CH₂—CH₂—, —CH₂—CH₂—CH₂—CH₂—, —CH═CH—CH═CH—, —CH₂—CH₂—CH₂—CH₂—CH₂—, —CH═CH—CH₂—CH₂—CH₂—, —CH₂—CH═CH—CH₂—CH₂—, —CH═CH—CH═CH—CH₂— or —CH═CH—CH═CH—CH═.

2. A non-therapeutic method of using the aromatic alkenoic acid derivatives of the formula I of claim 1
(i) for reducing appetite, and/or
(ii) for causing a feeling of satiation, and/or
(iii) as a mood enhancer.

3. The derivative of claim 1, wherein
more than 50% of the resulting double bond(s) are in E configuration,
R¹=methyl, ethyl
R²=hydrogen,
R³=2-methylpropyl, 2-methyl-1-butyl, or
R² and R³ together form a saturated carbocyclic ring consisting of —CH2-CH2-CH2-CH2-CH2-.

4. The derivative of claim 1, wherein said aromatic alkenoic acid derivative is selected from the group consisting of:
5-(1,3-benzodioxol-5-yl)-N-isobutylpent-2-enamide (compound 1),
7-(1,3-benzodioxol-5-yl)-N-isobutylhepta-2,4-dienamide (compound 2),
7-(1,3-benzodioxol-5-yl)-1-(1-piperidyl)hepta-2,4-dien-1-one (compound 3),
methyl 7-(1,3-benzodioxol-5-yl)hepta-2,4-dienoate (compound 4).

5. The derivative of claim 1, wherein more than 50% of the aromatic alkenoic acid derivatives are in the E configuration.

6. A method
(i) for non-therapeutic reduction of appetite, and/or
(ii) for non-therapeutically causing a feeling of satiation, and/or
(iii) for enhancing the mood,
comprising the step of:
administering an amount that (a) reduces the appetite and/or (b) causes a feeling of satiation and/or (c) enhances the mood of at least one aromatic alkenoic acid derivative compound of the formula I of claim 1.

7. A formulation comprising at least one aromatic alkenoic acid derivative of the formula I of claim 1
for use in a therapeutic method or in a non-therapeutic use,
(i) for reducing appetite, and/or
(ii) for causing a feeling of satiation, and/or
(iii) as a mood enhancer.

8. The formulation of claim 7, wherein
more than 50% of the resulting double bond(s) are in E configuration,
R¹=methyl, ethyl
R²=hydrogen,
R³=2-methylpropyl, 2-methyl-1-butyl, or
R² and R³ together form a saturated carbocyclic ring consisting of —CH2-CH2-CH2-CH2-CH2-, and
more than 50% of the aromatic alkenoic acid derivatives are in E configuration.

9. The formulation of claim 7, wherein the aromatic alkenoic acid derivative is selected from the group consisting of:
5-(1,3-benzodioxol-5-yl)-N-isobutylpent-2-enamide (compound 1),
7-(1,3-benzodioxol-5-yl)-N-isobutylhepta-2,4-dienamide (compound 2),
7-(1,3-benzodioxol-5-yl)-1-(1-piperidyl)hepta-2,4-dien-1-one (compound 3),
methyl 7-(1,3-benzodioxol-5-yl)hepta-2,4-dienoate (compound 4).

10. The formulation of claim 7, comprising N-isobutyl-2E,4E-decadienamide (trans-pellitorine) and/or nonivamide (N-nonanoylvanillylamine).

11. The formulation of claim 7, wherein the amount of aromatic alkenoic acid derivative(s) and/or N-isobutyl-2E,4E-decadienamide (trans-pellitorine) and/or nonivamide is used in a concentration which exhibits only a sub-threshold aroma value in the use concentration.

12. The formulation of claim 7, wherein the total concentration of the aromatic alkenoic acid derivative in the formulation is within a range of less than 20 mg/kg, based on the total mass of the formulation.

13. The formulation of claim 7, wherein the formulation is in the form of an orally consumable product or part of an orally consumable product.

14. The formulation of claim 7, wherein the formulation is selected from the group consisting of cosmetics, liquid or solid semi-luxury goods, foodstuffs, semi-finished goods, feedstuffs and medicaments.

15. An orally consumable product comprising at least one aromatic alkenoic acid derivative of the formula I of claim 1
wherein the aromatic alkenoic acid derivative is used in a concentration that reduces the appetite and/or causes a feeling of satiation.

16. The derivative of claim 4 which is methyl 7-(1,3-benzodioxol-5-yl)hepta-2,4-dienoate (compound 4) or a plant extract comprising methyl 7-(1,3-benzodioxol-5-yl)hepta-2,4-dienoate.
